(19) United States
(12) Patent Application Publication
(10) Pub. No.: US 2010/0159572 A1
(43) Pub. Date: Jun. 24, 2010

(54) DEVICE AND ASSOCIATED METHOD

(75) Inventors: Rui Chen, Clifton Park, NY (US); Anping Zhang, Rexford, NY (US); Anup Sood, Clifton Park, NY (US); Anthony John Murray, Lebanon, NJ (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 12/340,789

(22) Filed: Dec. 22, 2008

(51) Int. Cl. C12M 1/34; B01J 19/00; B05D 3/02

(52) U.S. Cl. 435/287.1; 422/68.1; 427/256

(57) ABSTRACT

A microfluidic device for detecting one or more molecules of interest comprising: a non-conductive substrate; wherein the non-conductive substrate is provided with a plurality of thermally active elements is provided. A method for selectively functionalizing a plurality of thermally active elements is also provided.

### FIELD OF INVENTION

The invention relates generally to methods and devices for detecting molecules of interest. One or more of the embodiments relate generally to microfluidic devices comprising selectively functionalized channels.

### BACKGROUND

Detection of chemical and biological analytes or molecules of interest may be required in various applications, for example, in pharmaceutical research, clinical diagnostics, food and beverage-quality monitoring, water purification, soil, water, and air-pollution monitoring, or in detection of chemical or biological warfare agents.

One or more chemical or biological analyte may be detected using molecules (probes) capable of specifically recognizing the analyte. Recognition may occur via highly specific interactions between two molecules, for example, an enzyme and a substrate, antibody and antigen, and the like. An occurrence or non-occurrence of the recognition reaction may be detected using suitable detection means as indication of the presence or absence of the analyte. In some applications, the analyte may be present in a very low concentration and the recognition event between the probe and the analyte may not be easily detected. Analyte amplification techniques may be employed to increase the concentration of the analyte, which may make it difficult to accurately quantify the analyte. In some analyte detection techniques, the recognition between the probe and the analyte may be partial or may not be completely specific resulting in false positives.

Recently, there has been an interest and active development in nanoscale sensors based on nanowires. Generally, nanoscale sensors are optimized for detecting specific species by specific preparation of the sensor surface, for example, by coating the surface with specific receptors. In particular, silicon nanowires have been employed as sensors for the detection of solution pH level, protein, gas molecules, DNA, cancer markers and neuronal signals. For detection of molecules at concentrations in the femtomolar range, non-specific binding of molecules to surfaces that are exposed to the fluid containing the analyte or molecule of interest becomes a significant consideration. Ideally the analyte of interest should bind only to the cognate binder on the nanowire. Binding of the analyte on other surfaces in the sensor has the undesired effect of reducing analyte mass sensitivity and sensor response. Development of multiplexed nanowire arrays is limited to some extent by the process of the device fabrication. The development is however limited by the process of locating different binders on a number of nanowires, and exclusively on those nanowires, for multiplex detection. Although attempts have been made to make a functional device using dec-9-enyl-carbamic acid tert-butyl ester to perform silicon-carbon specific functionalization, the yield obtained was poor. Polymers such as polytetrafluoroethylene (PTFE) coatings were also employed on the nanowire. The polymer coating was ablated by joule heating in order to reveal the nanowire for functionalization with an appropriate binding molecule by means of silane linkage chemistry. This approach is not suitable for modifying nanowire arrays with different binders due to the harsh conditions employed. For example, residual PTFE must be removed by an oxygen plasma prior to functionalization of nanowires by successive treatments.

Therefore there exists a need to have devices having selectively functionalized nanowires to improve the sensitivity and detection limit of the device. There also exists a need to have a method to selectively functionalize the nanowires without denaturing and/or destroying the functionalization.

### BRIEF DESCRIPTION

The methods and devices of the invention are designed to provide a device capable of being selectively functionalized at a plurality of thermally active elements and detecting one or more molecules of interest comprising: a non-conductive substrate; wherein the non-conductive substrate is provided with a plurality of thermally active elements. One or more of the embodiments of the device is adapted for use in a microfluidic device.

An example of a method of the invention, for selectively functionalizing a plurality of thermally active elements, comprises: providing a non-conductive substrate on which the plurality of thermally active elements are located. The method further comprises applying a material comprising an activatable functional group to at least a portion of one or more of the thermally active elements; and heating one or more of the thermally active element to a temperature sufficient to activate the activatable functional group.

### BRIEF DESCRIPTION OF DRAWING

These and other features, aspects and advantages of the present invention will become better understood when the following detailed description is read with reference to the accompanying figures.

FIG. 1 is a schematic illustration of a method for selectively functionalizing a plurality of thermally active elements according to one embodiment of the invention.

### DETAILED DESCRIPTION

To more clearly and concisely describe and point out the subject matter of the claimed invention, the following definitions are provided for specific terms, which are used in the following description and the appended claims. Throughout the specification, exemplification of specific terms should be considered as non-limiting examples. The precise use, choice of reagents, choice of variables such as concentration, volume, incubation time, incubation temperature, and the like may depend in large part on the particular application for which it is intended. It is to be understood that one of skill in the art will be able to identify suitable variables based on the present disclosure. It will be within the ability of those skilled in the art, however, given the benefit of this disclosure, to select and optimize suitable conditions for using the methods in accordance with the principles of the present invention, suitable for these and other types of applications.

In the following specification, and the claims that follow, reference will be made to a number of terms that have the following meanings. The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts while still being considered free of the modified term.

As used herein, the term “binder” refers to a molecule that may bind to one or more targets in the biological sample. A binder may specifically bind to a target. Suitable binders may include one or more of natural or modified peptides, proteins (e.g., antibodies, affibodies, or aptamers), nucleic acids (e.g., polynucleotides, DNA, RNA, or aptamers); polysaccharides (e.g., lectins, sugars), lipids, enzymes, enzyme substrates or inhibitors, ligands, receptors, antigens, or haptens. A suitable binder may be selected depending on the sample to be analyzed and the targets available for detection. For example, a target in the sample may include a ligand and the binder may include a receptor or a target may include a receptor and the binder may include a ligand. Similarly, a target may include an antigen and the binder may include an antibody or antibody fragment or vice versa. In some embodiments, a target may include a nucleic acid and the binder may include a complementary nucleic acid. In some embodiments, both the target and the binder may include proteins capable of binding to each other.

As used herein, the term “antibody” refers to an immunoglobulin that specifically binds to and is thereby defined as complementary with a particular spatial and polar organization of another molecule. The antibody may be monoclonal or polyclonal and may be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM. Functional antibody fragments may include portions of an antibody capable of retaining binding at similar affinity to full-length antibody (for example, Fab, Fv and F(ab′)₂, or Fab′). In addition, aggregates, polymers, and conjugates of immunoglobulins or their fragments may be used where appropriate so long as binding affinity for a particular molecule is substantially maintained.

As used herein, the term “peptide” refers to a sequence of amino acids connected to each other by peptide bonds between the alpha amino and carboxyl groups of adjacent amino acids. The amino acids may be the standard amino acids or some other non standard amino acids. Some of the standard nonpolar (hydrophobic) amino acids include alanine (Ala), leucine (Leu), isoleucine (Ile), valine (Val), proline (Pro), phenylalanine (Phe), tryptophan (Trp) and methionine (Met). The polar neutral amino acids include glycine (Gly), serine (Ser), threonine (Thr), cysteine (Cys), tyrosine (Tyr), asparagine (Asn) and glutamine (Gln). The positively charged (basic) amino acids include arginine (Arg), lysine (Lys) and histidine (His). The negatively charged (acidic) amino acids include aspartic acid (Asp) and glutamic acid (Glu). The non standard amino acids may be formed in body, for example by posttranslational modification, some examples of such amino acids being selenocysteine and pyrolysine. The peptides may be of a variety of lengths, either in their neutral (uncharged) form or in forms such as their salts. The peptides may be either free of modifications such as glycosylations, side chain oxidation or phosphorylation or comprising such modifications. Substitutes for an amino acid within the sequence may also be selected from other members of the class to which the amino acid belongs. A suitable peptide may also include peptides modified by additional substituents attached to the amino side chains, such as glycosyl units, lipids or inorganic ions such as phosphates as well as chemical modifications of the chains. Thus, the term “peptide” or its equivalent may be intended to include the appropriate amino acid sequence referenced, subject to the foregoing modifications, which do not destroy its functionality.

As used herein, the term “nucleotide” refers to both natural and modified nucleoside phosphates. The term “nucleoside” refers to a compound having a purine, deazapurine, pyrimidine or a modified base linked at the 1′ position or at an equivalent position to a sugar or a sugar substitute (e.g., a carbocyclic or an acyclic moiety). The nucleoside may contain a 2′-deoxy, 2′-hydroxyl or 2′,3′-dideoxy forms of sugar or sugar substitute as well as other substituted forms. The sugar moiety in the nucleoside phosphate may be a pentose sugar, such as ribose, and the phosphate esterification site may correspond to the hydroxyl group attached to the C-5 position of the pentose sugar of the nucleoside. A nucleotide may be, but is not limited to, a deoxyribonucleoside triphosphate (dNTP). Deoxyribonucleoside triphosphate may be, but is not limited to, a deoxyriboadenosine triphosphate (2′-deoxyadenosine 5′-triphosphate or dATP), a deoxyribocytosine triphosphate (2′-deoxycytidine 5′-triphosphate or dCTP), a deoxyriboguanosine triphosphate (2′-deoxyguanosine 5′-triphosphate or dGTP) or a deoxyribothymidine triphosphate (2′-deoxythymidine 5′-triphosphate or dTTP).

The term “oligonucleotide”, as used herein, refers to oligomers of nucleotides or derivatives thereof. Throughout the specification, whenever an oligonucleotide is represented by a sequence of letters, the nucleotides are in 5′→3′ order from left to right. In the letter sequence, letter A denotes adenosine, C denotes cytosine, G denotes guanosine, T denotes thymidine, W denotes A or T, and S denotes G or C. N represents a random nucleic acid base (e.g., N may be any of A, C, G, U, or T). A synthetic, locked, random nucleotide is represented by +N and a phosphorothioate modified random nucleotide is represented by *N.

“Nucleic acid,” or “oligonucleotide”, as used herein, may be a DNA, or a RNA, or its analogue (e.g., phosphorothioate analog). Nucleic acids or oligonucleotides may also include modified bases, backbones, and/or ends. Non-limiting examples of synthetic backbones include phosphorothioate, peptide nucleic acid, locked nucleic acid, xylose nucleic acid, or analogs thereof that confer stability and/or other advantages to the nucleic acids.

As used herein, the term “enzyme” refers to a protein molecule that can catalyze a chemical reaction of a substrate. In some embodiments, a suitable enzyme catalyzes a chemical reaction of the substrate to form a reaction product that can bind to a receptor (e.g., phenolic groups) present in the sample or a solid support to which the sample is bound. A receptor may be exogeneous (that is, a receptor extrinsically adhered to the sample or the solid-support) or endogeneous (receptors present intrinsically in the sample or the solid-support). Examples of suitable enzymes include peroxidases, oxidases, phosphatases, esterases, and glycosidases. Specific examples of suitable enzymes include horseradish peroxidase, alkaline phosphatase, β-D-galactosidase, lipase, and glucose oxidase.

As used herein, the term “biological sample” refers to a sample obtained from a biological subject, including samples of biological tissue or fluid origin obtained in vivo or in vitro. Such samples can be, but are not limited to, body fluid (e.g., blood, blood plasma, serum, or urine), organs, tissues, fractions, and cells isolated from mammals including, humans. Biological samples also may include sections of the biological sample including tissues (e.g., sectional portions of an organ or tissue). Biological samples may also include extracts from a biological sample, for example, an antigen from a biological fluid (e.g., blood or urine).

One or more embodiments are directed to a microfluidic device for detecting one or more molecules of interest. The microfluidic device comprises a non-conductive substrate; wherein the non-conductive substrate is provided with a plurality of thermally active elements.

In some embodiments, the non-conductive substrate comprises silicon, silicon wafer, glass, quartz, ZnO, TiO, carbon, or carbon nanotubes. In one example embodiment, the non-conductive substrate comprises silicon. In another example embodiment, the non-conductive substrate comprises a silicon wafer. In yet another example embodiment, the substrate comprises a silicon coated with silicon dioxide.

In some embodiments, the non-conductive substrate includes a plurality of thermally active elements, attached to the substrate so that the surface area within a certain “footprint” of the substrate is increased relative to the surface area within the same footprint without the plurality of thermally active elements. In one embodiment, the plurality of thermally active elements include at least one selected from silicon, glass, quartz, plastic, metal, polymers, TiO, ZnO, ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, GaN, GaP, GaAs, GaSb, InN, InP, InAs, InSb, PbS, PbSe, PbTe, AlS, AlP, AlSb, SiO₁, SiO₂, silicon carbide, silicon nitride, polyacrylonitrile (PAN), polymethylmethacrylate (PMMA), polydimethylsiloxane (PDMS), poly(ethylene terephthalate) (PETG), polyaniline, metal-organic polymers, polycarbonate, organic polymers, polyetherketone, polyimide, aromatic polymers, aliphatic polymers, polyvinyl alcohol, polystyrene, polyester, or polyamide. In some embodiments, the thermally active element comprises a nanowire. In some embodiments, the nanowire can include the same material as one or more substrate surface to which the nanowires are attached or associated. In certain other embodiments, the nanowires include a different material than the substrate surface.

The term “nanowire” as used herein, refers to a nanostructure typically characterized by at least one physical dimension less than about 1000 nm, less than about 500 nm, less than about 250 nm, less than about 150 nm, less than about 100 nm, less than about 50 nm, less than about 25 nm or even less than about 10 nm or 5 nm. In some embodiments, nanowires can typically have an aspect ratio greater than one, an aspect ratio of 2 or greater, an aspect ratio greater than about 10, an aspect ratio greater than about 20, or an aspect ratio greater than about 100, 200, 500, 1000, or 2000. In certain embodiments, the non conductive substrate comprises a nanowire having a diameter in a range from about 0.5 nm to about 300 nm. In some embodiments, the nanowires can have a substantially uniform diameter. In some embodiments, the diameter shows a variance less than about 20%, less than about 10%, less than about 5%, or less than about 1% over the region of greatest variability. In yet other embodiments, the nanowires can have a non-uniform diameter (i.e., they vary in diameter along their length). For example, a wide range of diameters could be desirable due to cost considerations and/or to create a more random surface. Also in certain embodiments, the nanowires of this invention are substantially crystalline and/or substantially monocrystalline.

The term nanowire optionally includes such structures as, e.g., nanofibers, nanoribbon, nanowhiskers, semi-conducting nanofibers, carbon and/or boron nanotubes or nanotubules and the like. Also, nanostructures having smaller aspect ratios (e.g., than those described above), such as nanorods, nanotetrapods, nanoposts and the like are also optionally included within the nanowire definition herein (in certain embodiments). In one example embodiment, the nanowires are individual nanowires. As used herein, “individual nanowires” means a nanowire free of contact with another nanowire (but not excluding contact of a type that may be desired between individual nanowires in a crossbar array). For example, typical individual nanowire can have a thickness as small as about 0.5 nm.

In one embodiment, at least a portion of the thermally active elements is contacted with a material comprising an activatable functional group. In some embodiments, the material comprising the activatable functional group is a masked silane. In some embodiments, the masked silane includes structural units derived from blocked isocyanate silane, at least one protection group selected from phenols, pyridinols, thiophenols, mercaptopyridines, mercaptans, bisulfite, oximes, amides, imides, imidazoles, amidines, or pyrazoles, a silyl carbamate, a silyl ester. In some embodiments, the masked silane includes structural units derived from alkylsiloxane, amino substituted alkoxy silane such as aminopropyltriethoxy silane, a carbamate functionalized silane such as 3-(triethoxysilylpropyl)-t-butyl carbamate. In some embodiments, the activatable functional group may be an ester such as for example 1-ethoxyethyl ester of carboxylic acid.

In one embodiment, at least a portion of the thermally active elements in contact with the masked silane are heated to a temperature less than about 200° C. In some embodiments, at least a portion of the plurality of thermally active elements in contact with the masked silane is heated to a temperature in a range from about 60° C. to about 160° C. In another embodiment, at least a portion of the thermally active elements in contact with the masked silane is heated to a temperature in a range from about 160° C. to about 200° C. In yet another embodiment, at least a portion of the thermally active elements in contact with the masked silane is heated to a temperature in a range from about 60° C. to about 100° C. In some embodiments, the heating of the thermally active elements unmasks the masked silane.

In one embodiment, the masked silane is adapted to be unmasked when exposed to a reactive group (also known herein after as “binder” are used interchangeably) to provide one or more thermally active elements that are functionalized to bind to one or more molecule of interest. In one example embodiment, the reactive group may be organic or inorganic in nature. Suitable organic ligands may include, but are not limited to, one or more of porphyrin, acetylacetonate, ethylenediaminetetracetate (EDTA), pyridine, bipyridine, terpyridine, ethylenediamine, oxalate, and the like. A suitable inorganic chemical probe may include, but is not limited to, an inorganic ligand, a metal complex, a metal salt, a nanocrystal, a nanoparticle, or combinations thereof. Suitable inorganic ligands may include, but are not limited to, one or more of halide, azide, ammonia, triphenylphosphine, thiocyanate, isothiocyanate, and the like. In some embodiments, the reactive group is a biological molecule that is capable of binding to the molecule of interest. A biological molecule may refer to a molecule obtained from a biological subject in vivo or in vitro. Non-limiting examples of biological molecules may include one or more of natural or modified peptides, proteins (e.g., antibodies, affibodies, or aptamers), nucleic acids (e.g., polynucleotides, DNA, RNA, or aptamers); polysaccharides (e.g., lectins, sugars), lipids, enzymes, enzyme substrates or inhibitors, antigens, haptens, vitamins, and the like. In some embodiments, the reactive group is at least one selected from —OH, —CHO, —COOH, —SO₃H, —CN, —NH₂, —SH, —COSH, —COOR, —NCS, —NCO, —NHS ester, -malemide, aziridine, -sulfonyl chloride, -epoxide, disulfide, a halide, a nucleic acid, an antibody, an antigen, a sugar, a carbohydrate, an amino acid, a protein, or an enzyme.

An example of a method of making the device comprises, providing a wafer comprising a silicon substrate on which a plurality of thermally active elements are located; and applying a material comprising an activatable functional group to at least a portion of the silicon substrate comprising one or more of the thermally active elements. In some embodiments, at least a portion of the activated elements can be functionalized with binders for one or more of the molecules of interest.

In some embodiments, the plurality of thermally active elements are activated individually. In some embodiments, the plurality of thermally active elements are activated simultaneously. In some embodiments the plurality of thermally active elements on the device may be simultaneously activated, for example, multiple devices may be functionalized at the wafer scale prior to dicing into individual devices. In some embodiments, the plurality of thermally active elements are activated serially. In some embodiments, the plurality of thermally active elements are activated selectively.

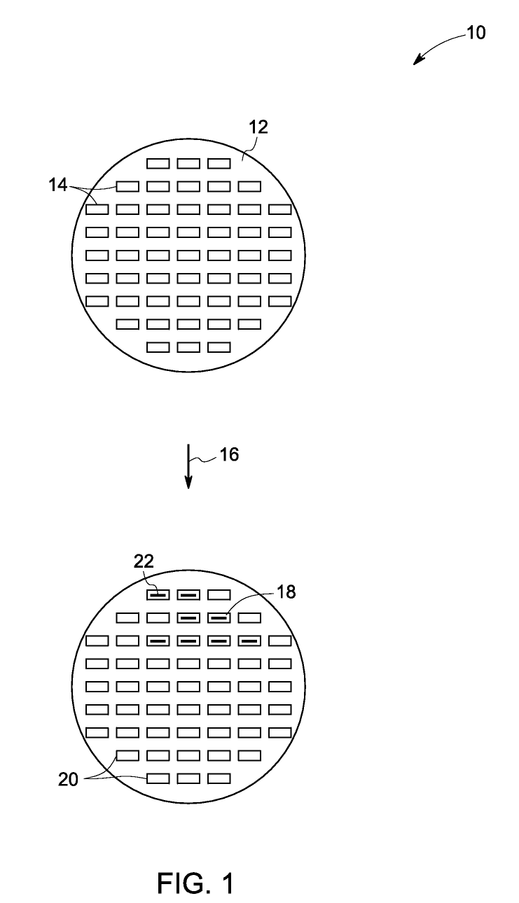

In some embodiments, the method 10 depicted in FIG. 1, comprises, providing a silicon substrate like for example a silicon wafer (12). The wafer (12) comprises a plurality of thermally active elements (14). In some embodiments, the functionalization is a wafer level functionalization (16). A material comprising an activatable functional group, is applied to at least a portion of the wafer (18). Then a binder material is applied to at least a portion of the wafer that contains the material comprising the activatable functional group (22). The unfunctionalized thermally active element (20) is then treated with another binder material and the process may be repeated.

In some embodiments, after activation, the binders may be added to be in contact with at least a portion of the thermally activated elements. In one embodiment, microfluidic devices may be used. In one embodiment, the microfluidic device for detecting one or more molecules of interest includes a non-conductive substrate provided with a plurality of thermally active elements, wherein at least a portion of the thermally active elements are in contact with a binder for the molecule of interest. In some embodiments, the thermally active element is capable of binding to the binder by a non-covalent bond formation. In some other embodiments, the thermally active element is capable of binding to the binder by a covalent bond formation.

In some embodiments, the molecule of interest may bind to the thermally activated elements that are functionalized with a binder in a specific manner. As used herein, the term “bind” may refer to the specific recognition of one of two different molecules for the other compared to substantially less recognition of other molecules. For example, a protein-specific molecule that may bind to a protein as a molecule of interest. Examples of suitable protein-specific molecules may include antibodies and antibody fragments, nucleic acids (for example, aptamers that recognize protein analytes), or protein substrates. In some embodiments, a molecule of interest may include an antigen and the probe(s) may include an antibody. A suitable antibody may include monoclonal antibodies, polyclonal antibodies, multispecific antibodies (for example, bispecific antibodies), or antibody fragments so long as they bind specifically to an antigen molecule of interest. In the case of specific detection of an analyte, a binder for that analyte is attached to the nanowire. In one embodiment, the analyte, on binding to binder on the nanowire, may change the charge, the charge density or distribution of charge on the nanowire surface and thereby changing the conductance of the nanowire. In some embodiment, the conductance of the nanowire may be used as a means to measure the concentration of the analyte. In one example embodiment, the sensitivity may be greatest for those analytes that may create not only a large change in charge, charge density, or charge distribution, but also when those changes are close to the surface of the nanowire and when the binder has a high affinity for the analyte.

The term “molecule of interest” or “analyte” are used interchangeably. In some embodiments, the molecule of interest can be determined by the type and nature of analysis required for the sample. In some embodiments, the analysis can provide information about the presence or absence of a molecule of interest in the sample. In another embodiment, an analysis can provide information on a state of a sample. For example, if the sample includes a drinking water sample, the analysis may provide information about the concentration of bacteria in the sample and thus the potability of the sample. Similarly, if the sample includes a tissue sample, the methods disclosed herein can be used to detect molecule(s) of interest that can help in comparing different types of cells or tissues, comparing different developmental stages, detecting the presence of a disease or abnormality, determining the type of disease abnormality or investigating the interactions between multiple molecules of interest.

In one embodiment, the molecule of interest may include one or more biological agents. Suitable biological agents may include pathogens, toxins, or combinations thereof. Biological agents may also include prions, microorganisms (viruses, bacteria and fungi) and some unicellular and multicellular eukaryotes (for example parasites) and their associated toxins. Pathogens are infectious agents that can cause disease or illness to their host (animal or plant). Pathogens may include one or more of bacteria, viruses, protozoa, fungi, parasites, or prions.

The molecule of interest can be living or nonliving in nature. In one embodiment, the molecule of interest can include a chemical warfare agent. In one embodiment, the molecule of interest is a pollutant, such as one or more of air pollutants, soil pollutants, or water pollutants.

In some embodiments, the molecules of interest are at concentrations in a sample in a range from about 1 fM to about 1 mM. In one example embodiment, the plurality of thermally active elements are functionalized to bind to one or more of the molecules of interest.

In some embodiments, the microfluidic device includes one or more functionalized thermally active elements to detect the presence or absence of a plurality of one or more molecules of interest. In one embodiment, the plurality of thermally active elements include one or more nanowires. In some embodiments, the one or more nanowires can be oriented randomly, parallel to one another or in an array on a substrate. The plurality of thermally active elements comprising nanowires can be differentially functionalized as described above, thereby varying the sensitivity of each nanowire to the molecule of interest. Alternatively, individual nanowires present in the plurality of thermally active element can be selected based on their ability to interact with specific molecules of interest, thereby allowing the detection of a variety of molecules of interest.

The molecule of interest can be living or nonliving in nature. In one embodiment, the molecule of interest is a pollutant, such as one or more of air pollutants, soil pollutants, or water pollutants. Air pollutants can include one or more of carbon monoxide, sulfur dioxide, chlorofluorocarbon, or nitrogen dioxide. Suitable soil pollutants can include one or more of hydrocarbon, heavy metal, herbicide, pesticide, or chlorinated hydrocarbon. Suitable water pollutants can include one or more heavy metals, fertilizers, herbicides, insecticides, or pathogens. In one embodiment, the molecule of interest includes one or more of phosphate, molybdate, magnesium, sulfite, or calcium. In one embodiment, the molecule of interest can include one or more spoilage indicators.

In one embodiment, the molecule of interest that can be detected using the compositions disclosed herein, can include one or more biomolecules. In one embodiment, a biomolecule-based molecule of interest can be part of a biological agent, such as, a pathogen. In one embodiment, a biomolecule can be used for diagnostic, therapeutic, or prognostic applications, for example, in RNA or DNA assays. Suitable biomolecules can include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, vitamins, antigens, or haptens. The term “molecule of interest” refers to both separate molecules and to portions of such molecules, such as an epitope of a protein, that can bind specifically with one or more probes.

In some embodiments, the sample containing the molecule of interest can be in a region exposed to the plurality of thermally active element that is functionalized, wherein a sample in the sample exposure region addresses at least a portion of the plurality of thermally active element that is functionalized. Examples of sample exposure regions include, but are not limited to, a well, a channel, a microchannel, and a gel. In one example embodiment, the sample exposure region holds a sample proximate to the thermally activated element comprising the nanowire, which is functionalised, or can direct a sample toward the functionalized nanowire for determination of the molecule of interest in the sample. The functionalized nanowire can be positioned adjacent to or within the sample exposure region. In some embodiments, the functionalized nanowire can be a probe that is inserted into a fluid or fluid flow path. The nanowire probe can also comprise a micro-needle and the sample exposure region can be addressable by a biological sample.

In some embodiments, chemical changes associated with the plurality of thermally active element can modulate the properties of the plurality of thermally active element comprising the nanowire. The interaction of the plurality of thermally active element comprising the nanowire with the binder and with the molecule of interest can change the property such as electronic, optical, or the like. In some embodiments, the interaction of the plurality of thermally active element comprising the nanowire with the binder and with the molecule of interest can change the electric property of the Where a detector is present, any detector capable of determining a property associated with the nanowire can be used. An electronic property of the nanowire can be, for example, its conductivity, resistivity, etc. An optical property associated with the nanowire can include its emission intensity, or emission wavelength where the nanowire is an emissive nanowire where emission occurs. For example, the detector can be constructed for measuring a change in an electronic or magnetic property (e.g. voltage, current, conductivity, resistance, impedance, inductance, charge, etc.), or fluorescence can be used. In some embodiments, the reactive group is a fluorophore, which can be excited by exposure to a particular wavelength of light, which would change on interaction with the molecule of interest. In one embodiment, the analyte, on binding to binder on the nanowire, may change the charge, the charge density or distribution of charge on the nanowire surface and thereby changing the conductance of the nanowire.

In some embodiments, the plurality of thermally active elements comprising the nanowire may be positioned in a microfluidic channel. One or more different nanowires may cross the same microchannel at different positions to detect a different molecule of interest or to measure flow rate of the same molecule of interest. In another embodiment, one or more nanowires positioned in a microfluidic channel may form one of the analytic elements in a microarray for a cassette or a lab on a chip device. Those skilled in the art would know such cassette or lab on a chip device will be in particular suitable for high throughout chemical or biological analysis and combinational drug discovery. The ability to include multiple nanowires in one nanoscale sensor, also allows for the simultaneous detection of different molecules of interest suspected of being present in a single sample.

### EXAMPLES

Unless specified otherwise, ingredients described in the examples are commercially available from common chemical suppliers. Some abbreviations used in the examples section are expanded as follows: “mg”: milligrams; “ng”: nanograms; “pg”: picograms; “fm” femtomole, “fg”: femtograms; “mL”: milliliters; “mg/mL”: milligrams per milliliter; “mM”: millimolar; “mmol”: millimoles; “pM”: picomolar; “pmol”: picomoles; “μL”: microliters; “min.”: minutes and “h.”: hours.

Masked Silane Treatment

A solution of about 2% (3-triethoxysilylpropyl)-t-butylcarbamate, procured from Gelest (25 grams), was prepared in ethanol (about 10 ml) in the presence of 0.1%-0.3% acetic acid (about 0.01-0.03 ml). The silicon wafer was cleaned with oxygen plasma in a Harrick Plasma Cleaner PDC-32G, (Harrick Plasma, Ithaca, N.Y.) for about 10 minutes. After cleaning, the silicon wafer was immersed in the (3-triethoxysilylpropyl)-t-butylcarbamate solution overnight. At the end of the stipulated time, the wafer was rinsed with ethanol (about 50 ml) and blown dry in air. The nature of silane modified silicon surfaces (whether they are hydrophilic or hydrophobic) was determined by measuring the contact angle before and after coating.

The contact angle was measured by employing the VCA-Optima instrument from AST Products, Inc; Massachusetts US). The contact angle was measured to determine the hydrophilic or hydrophobic nature of the silicon wafer surfaces. The contact angle data is given in Table 1.

Table 1: Contact Angle Data with Silianization and Sonication Treatment.

TABLE 1
Contact Angle Data with silianization and Sonication treatment.

| | CEx. 1 | Ex. 1 | Ex. 2 | Ex. 3 |
|---|---|---|---|---|
| Before sonication (0 minutes) | 4 | 56.85 | 75.65 | 58.53 |
| Sonication Time | — | — | 72.45 | 53.47 |
| (2 minutes) | | | | |
| Sonication time | — | — | 67.52 | 53.5 |
| (12 minutes) | | | | |

As shown in Table 1, the comparative example (CEx. 1, which is the bare silicon wafer SiO2 layer) is more hydrophilic at the contact angle 4 (i.e. less than 50). Also, the contact angle for the t-butyl masked silane (with 0.3% acetic acid) coated wafer Ex. 2, is greater than the contact angle for the t-butyl masked silane (with 0.1% acetic acid, Ex. 1) coated wafer Ex. 1, which indicates that the wafer is more hydrophobic and better coated in Ex. 2. Sonication in ethanol was carried out for Ex. 2 and Ex. 3, which is an aminetrimethoxy silane coated wafer. Sonication was employed to remove any adsorbed silane reagents. A slight decrease in the contact angle was noticed when the sonication was carried out for 12 minutes.

Heat Unmasking, and Surface Amine Group Analysis

A laboratory hotplate equipped with a thermocouple probe was used to heat silanized wafers at certain temperature in a range from about 160° C. to about 200° C. for predetermined intervals of about 1 to about 5 minutes. Following this the wafers were cooled and cleaned with water. In some cases, a digital hotplate was adopted to better control the heating temperature within 1° C. Their contact angle changes after heating were measured.

Table 2: Contact Angle Data with Heat Unmasking Treatment.

TABLE 2
Contact Angle Data with heat unmasking treatment.

| | | Ex. 2 | | | |
|---|---|---|---|---|---|
| | | (t-butyl | Ex. 3 | | |
| Heating | | masked | (amine trimethoxy | CEx. 2 | CEx. 3 |
| (° C., | | silane coated | silane coated | (Superamine | (Superaldehyde |
| minutes) | CEx. 1 | wafer) | wafer) | slide) | slide) |
| | 4 | 77 | 38.23 | 58 | 56.87 |
| 160° C., 1 min | — | 38.56 | — | — | — |
| 160° C., 2 min | — | 30.15 | — | — | — |
| 160° C., 3 min | — | 25.48 | — | — | — |
| 180° C., 2 min | — | 18.97 | — | — | — |
| 230° C., | — | 16.65 | 28.87 | — | — |
| 2 min | | | | | |
| 230° C., 5 min | — | 7.17 | 36.87 | — | — |

As shown in Table 2, the change in the functional group during the heat unmasking process results in a change of the contact angle of the wafer. It is presumed that the isocyanate or amine groups are more hydrophilic than a t-butyl masking group. Table 2 measures the contact angle of the wafer after heating at 160° C., 180° C. and 230° C. for about 1 to about 5 minutes. The hydrophobicity is found to decrease as the heating continues at 180° C. as indicated by a decrease in the contact angle.

A fluorogenic dye (3-2-(furoyl) quinoline-2-carboxaldehyde), (ATTO-TAG FQ) procured from Invitrogen Corporation, California, USA was used to investigate surface functional group on heated unmasked wafer. About 50 μL of 10 mM ATTO-TAG FQ) stock solution was mixed with 50 μL 200 mM potassium cyanide and 400 μL borate buffer (100 mM borate buffer, pH 9, from Sigma Aldrich) to make a working solution. This working solution was then applied onto the surface of silicon wafers and was incubated for about 1 hour. A Superamine slide (from Arrayit.com, hereinafter known as “Control 2”) was treated with the working solution and was incubated for about 1 hour. The silicon wafers were then washed with water. The washed silicon wafers were imaged using a Typhoon 9400 imager (GE Healthcare, Milwaukee, USA). The excitation was set at about 488 nm, and the emission at about 580 nm with a bandwidth of 30 nm, were measured with a photomultiplier tube setting of 600V. ImageQuant software was used to analyze the fluorescence images.

The “control 2” was also treated in the above manner and the imaged using Typhoon 9400 imager.

On heating, the isocyanate group was unmasked, and when exposed to moisture, the isocyanate group decomposed to form an amine group and carbon dioxide (CO₂). To study the surface NH₂ group, the fluorogenic dye ATTO FQ was used. The FQ dye is intrinsically nonfluorescent but reacts rapidly with primary amines to yield a fluorescent derivative. CEx. 2 (commercial Superamine slide) and example 1, which is the masked isocyanate silane treated wafers (heat unmask at various conditions and non-heat treated wafer as control) were compared. The amine groups were detected for the masked silane coated wafers (Ex. 1) after heating at 180° C. for 4 min, and their amine levels were found to be comparable to the Superamine slide (CEx. 2). Also, when Ex. 1 was not heated, the fluorescence was less (values shown in Table 3)

Table 3

TABLE 3

| | FQ fluorescence (relative to the amine conc. |
|---|---|
| Sample | on the surface of the wafer) |
| CEx. 2 | 2143.84 |
| Ex. 1 (heated to 180° C., 4 | 2249.96 |
| minutes) | |
| Ex. 1 (not heated) | 851.15 |

### Antibody Immobilization and Detection

The silane treated wafers and the heat-treated silane wafers, prepared using the method described above, were treated with about 2.5% glutaraldehyde solution for about 1 hour. Following the glutaraldehyde treatment, the wafers were rinsed with ethanol (about 50 ml). About 1 μL 6 mg/ml insulin antibody was added to about 56 μL of the phosphate buffer (10 mM, pH 8.4). Following the addition, about 3 μL sodium cyanoborohydride (40 mM) was added to the insulin antibody solution above to make 0.1 mg/mL solution. The resultant solution was added on top of the silane treated wafer. The treated silane wafers were heated and incubated in humidity chamber for about 1 hour. At the end of the stipulated time, the wafers were rinsed with the phosphate buffer solution (about 50 mL) containing about 0.1% Tween-20 FITC-insulin (Fluorescein labeled insulin) about 0.02 mg per mL was added to the wafers and the wafers were further incubated for about 1 hour. At the end of about 1 hour, the wafers were rinsed with phosphate buffer solution (about 50 mL) containing about 0.1% Tween-20. The wafers thus treated were then imaged using a Typhoon 9400 imager (GE Healthcare, Milwaukee, USA) with a setting for FITC fluorescence (fluorescence channel of 488 nm excitation, 520BP40 emission). The excitation was set at about 488 nm, and the emission at about 520 nm with a bandwidth of 40 nm, were measured with a photomultiplier tube setting of 600 V. An ImageQuant software was used to analyze the fluorescence images.

The “control 2” was also treated with about 2.5% glutaraldehyde solution as mentioned in the above procedure and the imaged using Typhoon 9400 imager.

As shown in Table 4, in Ex. 4 and Ex. 5, the fluorescence signal is greater than the non-spotted regions as a result of FITC-insulin binding to surface Ab due to the selective functionalization of the wafer. The control wafer, which was the masked silane coated wafer without heat treatment (CEx. 4), had some fluorescence signal above background, likely due to the non-specific adsorption of Ab by hydrophobic t-butyl masking group.

TABLE 4

| | Fluorescence | |
|---|---|---|
| | signal on | Background |
| Sample | spot | signal |
| Ex. 4: masked silane treated wafer heat | 6500 | 4000 |
| at 160° C. for 2 min, treated with | | |
| glutaraldehyde and Ab then block and | | |
| add FITC-insulin | | |
| Ex. 5: masked silane treated wafer heat | 7600 | 4500 |
| at 160° C. for 4 min, treated with | | |
| glutaraldehyde and Ab then block and | | |
| add FITC-insulin | | |
| CEx. 4: control: masked silane treated | 5500 | 5000 |
| wafer treated with glutaraldehyde and | | |
| Ab then block and add FITC-insulin | | |

The foregoing examples are illustrative of some features of the invention, and are selected embodiments from a manifold of all possible embodiments. The invention may be embodied in other specific forms without departing from the spirit or essential characteristics thereof. While only certain features of the invention have been illustrated and described herein, one skilled in the art, given the benefit of this disclosure, will be able to make modifications/changes to optimize the parameters. The foregoing embodiments are therefore to be considered in all respects as illustrative rather than limiting on the invention described herein. Where necessary, ranges have been supplied, and those ranges are inclusive of all sub-ranges there between.

1. A microfluidic device for detecting one or more molecules of interest comprising:
a non-conductive substrate;
wherein the non-conductive substrate is provided with a plurality of thermally active elements.

2. The device of claim 1, wherein the thermally active element comprises a nanowire.

3. The device of claim 2, wherein the non-conductive substrate comprises silicon.

4. The device of claim 2, wherein the non-conductive substrate is a silicon wafer.

5. The device of claim 1, wherein at least a portion of the thermally active element is contacted with a material comprising an activatable functional group.

6. The device of claim 1, wherein the activatable functional group is a masked silane.

7. The device of claim 6, wherein the masked silane comprises structural units derived from t butyl isocyanate, at least one protection group selected from phenols, pyridinols, thiophenols, mercaptopyridines, mercaptans, bisulfite, oximes, ester, amides, imides, imidazoles, amidines, or pyrazoles, a silyl carbamate, a silyl ester.

8. The device of claim 6, wherein the masked silane is adapted to be unmasked when heated to a temperature in a range from about 60° C. to about 200° C.

9. The device of claim 1, wherein at least a portion of the thermally activated element is functionalized with a binder.

10. The device of claim 9, wherein the binder is at least one selected from —OH, —CHO, —COOH, —SO₃H, —CN, —NH₂, —SH, —COSH, —COOR, NCS, —NCO, —NHS ester, -malemide, aziridine, -sulfonyl chloride, -epoxide, disulfide, a halide, a nucleic acid, an antibody, an antigen, a sugar, a carbohydrate, an amino acid, a protein, or an enzyme.

11. The device of claim 1, wherein one or more of the thermally active elements are activated individually.

12. The device of claim 1, wherein a plurality of the thermally active elements are activated simultaneously.

13. The device of claim 1, wherein the non conductive substrate comprises a nanowire having a diameter in a range from about 0.5 nm to about 300 nm.

14. The device of claim 1 wherein one or more of the molecules of interest are at concentrations in a sample in a range from about 1 fM to about 1 mM, wherein one or more of the thermally active elements are functionalized to bind to one or more of the molecules of interest.

15. A method of making the device of claim 1, comprising,
providing a wafer comprising a silicon substrate on which a plurality of thermally active elements are located;
applying a material comprising an activatable functional group to at least a portion of the silicon substrate comprising one or more of the thermally active elements; and
heating one or more of the thermally active elements to a temperature sufficient to activate the activatable functional group.

16. A method for selectively functionalizing a plurality of thermally active elements comprising:
providing a non-conductive substrate on which the plurality of thermally active elements are located;
applying a material comprising an activatable functional group to at least a portion of the substrate comprising one or more of the thermally active elements; and
heating one or more of the thermally active elements to a temperature sufficient to activate the activatable functional group.

17. The method of claim 16, wherein the activatable functional group is a masked silane.

18. The method of claim 16, wherein one or more of the thermally active elements are heated to a temperature in a range from about 60° C. to about 200° C.

19. The method of claim 16, further comprising selectively binding one or more molecules of interest to at least a portion of the thermally active element functionalized by a binder.
